(19) United States
(12) Patent Application Publication
(10) Pub. No.: US 2024/0033163 A1
(43) Pub. Date: Feb. 1, 2024

(54) FLUID PRESSURE CONTROL SYSTEM, CONNECTOR AND COUPLING ASSEMBLY

(71) Applicant: Arjo IP Holding Aktiebolag, Malmö (SE)

(72) Inventor: Michael David Newton, San Antonio, TX (US)

(73) Assignee: Arjo IP Holding Aktiebolag, Malmö (SE)

(21) Appl. No.: 18/267,908

(22) PCT Filed: Dec. 15, 2021
(86) PCT No.: PCT/SE2021/051262

§ 371 Date: Jun. 16, 2023

(30) Foreign Application Priority Data
Dec. 18, 2020 (SE) 2051490-7

(51) Int. Cl. A61H 9/00

(52) U.S. Cl. CPC A61H 9/0092; F16L 2201/10; A61H 2201/1238; A61H 2201/1409; A61H 2201/5092

(57) ABSTRACT

Present invention relates to a coupling assembly (300, 600) for fluidically connecting an inflatable/deflatable article (120) and a pump (110) in a fluid pressure control system (100) and a fluid pressure control system (100) comprising such a coupling assembly (300, 600). The coupling assembly (300, 600) comprises a position indicating arrangement (380) which comprises a sensing arrangement (320) and a position indicating part (390, 490, 690).

### TECHNICAL FIELD

The present invention relates to a coupling assembly for fluidically connecting an inflatable/deflatable article in a fluid pressure control system. The present invention further relates to a fluid pressure control system comprising an inflatable/deflatable article, a pump and a controller. In addition, present invention relates to a method for monitoring a coupling assembly for fluidically connecting an inflatable/deflatable article and a pump in a fluid pressure control system as well as a pump system and inflatable/deflatable article arrangement.

### BACKGROUND

This invention relates to pneumatic systems and in particular to pneumatic systems having an inflatable/deflatable article connected to a fluid source, for example a pump.

It is known for such systems to have a coupling assembly connecting the article to the fluid source, the coupling assembly comprising a male insert and a cooperating female receptacle for receiving the male insert to form a coupled state. The male insert or female receptacle includes a mechanical latch cooperating with a cavity on a corresponding surface of the other for mechanically latching and unlatching the coupling assembly. The male insert member and the female receptacle both defines a pathway for the flow of fluid through it when in the coupled state. A seal member extends between the male insert member surface and the surface of the female receptacle to provide a fluid tight seal when in the coupled state.

Current coupling assemblies often utilizes a type of ‘quick-connect’ or ‘snap fit’ two-part connector arrangement. This involves a purely mechanical engagement in order to provide the fluid passage between the pump and the inflatable garment. This can involve one or more separate air paths. Many different connectors are available in this style and look very similar. It is therefore relatively easy for the user (or patient) to try to connect items that at first glance would appear to be compatible but which are not intended to operate together. As a result, there is a potential for a complication and hazard associated with the interconnection of these devices.

The relatively small physical size and shape of some of connectors commonly in use does not readily allow for extensive marking and physical features to aid the user to avoid misconnection, particularly those who may have vision limitations or limited dexterity such as those products designed for use in non-acute locations such as in a homecare environments. The use of color coding is also not fully effective for all users because of color vision deficiency (color blindness). The use of product marking techniques in general in itself also does not provide a failsafe operation as these can be ignored, inadvertently used/mis-used due to lack of understanding or coordination in the marketplace. The integration of the insertion monitoring and identification process with the underlying operation of the product therefore provides a more effective solution than marking. Thus, there is a need for systems which mitigate the risks of such complications and hazards.

This may be performed by the article or the connector of the article being provided with a specific identification component, as described in for example U.S. Pat. Nos. 7,398,803 and 10,675,210. This may also be performed by sensing of the characteristics of the air inflation process to detect a connected article type, such as a garment type, as described in for example U.S. Pat. Nos. 8,235,922 and 8,734,369.

Using the characteristics of the air inflation process is not always optimal due to limited ability to differentiate between differing inflatable/deflatable articles that share very similar inflatable volumes. It also limits the identification process by only allowing this detection to occur when the article is actually being inflated and hence prophylaxis is running. There is no sensing employed at any other time apart from during the time the article is in an inflating/inflated state, which results in a considerably slower and less responsive user experience. The feedback to the user is therefore slower and a user needs to wait up to a typical inflation cycle (between 40 seconds to 1 minute) to allow for the pump to update its measurement and confirm if any article is still attached, its type or if an article has been detached.

The characteristics based method may also be susceptible to variations in the case of the article being a garment due to for example garment fitting, the tightness to the limb or limb size affecting the inflation and hence the sensing accuracy. These variations can affect the accuracy of any garment detection method. Additionally, if there is a varying degree of inflation due to manufacturing tolerances in the air source then this adds further uncertainty and limits the accuracy of this approach. Further, if an equivalent size of a similar but non-approved inflatable garment is fitted by the user to a pump in error then the inflation characteristics could be very similar and hence errors be introduced. Therefore, this approach does not protect the user by preventing the use of 3r d party garments that may not be regulatory approved or tested as being compatible or which could potentially provide a clinical risk if used with the pump. This method also is at risk of failing if there is a change in the garment due to ageing of the inflatable chamber due to material changes, the effects of reprocessing or if there is a garment air leak due to device failure

Whilst some tactile, physical and visual identification features can be useful—a misconnection can still occur if the fluidic connection can be physically engaged, even a partial connection can occur if the controller/pump is automatically able to supply air to the article without some additional securing or enablement feature.

This partial connector insertion problem can also potentially result in a non-complete fluid connection and a temporary physical connection that can be subsequently and inadvertently disconnected during normal use. Whilst there is often a mechanical latch available on some connectors—it is necessary to manually fully engage the connector in order for the latch to be secured.

Additionally, in more recent years, there has been a rise in the use of reprocessing of articles, for example compression garments, as a means of reducing medical waste and device costs. This reprocessing results in the same article and connector often being re-used by many different patients with an associated reprocessing process applied between each use. Therefore, the connector has to operate for an increased number of operational insertion and removal cycles and an associated increased mechanical level of wear and tear, as a result connectors can sometimes become worn in use.

The nature of the reprocessing process itself can have an effect on the connector as it can involve the use of washing cycles with raised water temperatures as well as the use of specialist processes and chemicals intended to ensure the garment is disinfected between each patient use such as ethylene oxide (EtO) sterilization. The effect of the temperature and chemicals used in this type of reprocessing can have an adverse effect on the plastic components used in the garment and connector as well as impacting other non-plastic parts within the connector.

This reprocessing is often performed by separate 3rd party reprocessing companies whom are not the original equipment manufacturer or designer of the combined system, pump or the inflatable/deflatable article. As a result, in some cases, it is likely that the reprocessing company does not have access to the original manufacturer's data such as detailed design specifications, material performance data, test requirements or information relating to the connection and inflation mechanism between the article and the pump.

As a result, there can be an increase in the mechanical wear and tear on the article connector during this significantly extended product lifetime, it is therefore possible that the mechanical aspects of the connection can be adversely affected. This can result in a degradation in the performance of the connector resulting in reduced sealing, engagement and retention. Over the extended lifetime of use of a reprocessable article, the connection of the article to the pump can become impaired potentially resulting in potential product failure with the resulting risk of reduced prophylaxis being delivered to the patient.

In the light of the above, there is a need for a system which addresses and mitigates the risk for misconnections and partial connections as well as reduces the risk of product failure due to wear. There is further a need for a system which helps the user to identify a faulty or worn out connector or inflatable/deflatable article.

### SUMMARY

According to an aspect, a coupling assembly for fluidically connecting an inflatable/deflatable article and a pump in a fluid pressure control system. The coupling assembly comprising a connector and a connecting member. The connector is connectable to the connecting member to upon engagement form a fluid pathway through said connector and connecting member.

The coupling assembly further comprises a position indicating arrangement. Said position indicating arrangement comprises a position indicating part provided on the connector or the connecting member and a sensing arrangement for detecting the position of the position indicating part relative said sensing arrangement during engagement between said connector and connecting member.

According to an aspect, a pump system configured to be connected to an inflatable/deflatable article in a fluid pressure control system by means of a coupling assembly according to the above. The pump system comprises the pump and a controller operatively connected to the pump for controlling said pump and the sensing arrangement.

According to an aspect, a connector for a coupling assembly for fluidically connecting an inflatable/deflatable article and a pump in a fluid pressure control system is provided. The connector is connectable to the connecting member to upon engagement form a fluid pathway through said connector and connecting member by means of insertion of a distal part of the connector into the connecting member.

The distal part is movable inside the connecting member along a connection axis extending distally from the connector between a coupled position and a non-coupled position.

The connector is provided with a position indicating part, whereby the position of the position indicating part is detectable by means of a sensing arrangement during engagement between the connector and the connecting member.

According to an aspect, an inflatable/deflatable article arrangement for connection to a pump in a fluid pressure control system by means of a coupling assembly. The inflatable/deflatable article arrangement comprises an inflatable/deflatable article and a connector according to the above.

According to an aspect, a fluid pressure control system is provided. The fluid pressure control system comprises an inflatable/deflatable article, a pump and a controller operatively connected to the pump for controlling said pump.

The fluid pressure control system further comprises a coupling assembly for fluidically connecting the inflatable/deflatable article and the pump. The coupling assembly comprises a connector and a connecting member. The connector is connectable to the connecting member to upon engagement form a fluid pathway through said connector and connecting member.

The coupling assembly further comprises a position indicating arrangement. the position indicating arrangement comprises a position indicating part provided on the connector or the connecting member and a sensing arrangement for detecting the position of the position indicating part relative said sensing arrangement during engagement between said connector and connecting member.

According to an aspect, a method is provided for monitoring a coupling assembly for fluidically connecting an inflatable/deflatable article and a pump of a fluid pressure control system. The fluid pressure control system comprises a controller operatively connected to the pump for controlling said pump. The coupling assembly comprises a connector and a connecting member. The connector is connectable to the connecting member to upon engagement form a fluid pathway through said connector and connecting member by means of insertion of a distal part of the connector into the connecting member. The distal part is movable inside the connecting member along a connection axis extending distally from the connector between a coupled position and a non-coupled position. The coupling assembly comprises a position indicating arrangement comprising a position indicating part provided on the connector or the connecting member and a sensing arrangement.

The method comprises detecting the position of the position indicating part provided on the connector or the connecting member relative to the other of the connector or the connecting member by means of the sensing arrangement.

The method further comprises generating a position signal to the controller indicative of the position of the distal part relative to the connecting member. The position signal is based on the detected position of the position indicating part.

Further objects and features of the present invention will appear from the following detailed description of embodiments of the invention.

### BRIEF DESCRIPTION OF DRAWINGS

The invention will be described with reference to the accompanying drawings, in which:

FIG. 1 depicts a fluid pressure control system according to an embodiment of the present invention.

FIG. 2 depicts a fluid pressure control system and various position indicating parts according to an embodiment of the present invention.

FIG. 3 depicts a schematic diagram of the operation of a fluid pressure control system and coupling assembly according to an embodiment of the present invention.

FIG. 4 depicts a schematic system diagram of a fluid pressure control system according to an embodiment of the present invention.

FIG. 5a-e depicts the operation of a coupling assembly of a fluid pressure control system according to an embodiment of the present invention.

### DETAILED DESCRIPTION

A number of differing connector styles, types and sizes are used in healthcare to provide fluidic connection within a patient environment covering a range of air and liquids for a variety of applications. Some standards exist in the area of medical connectors for certain medical applications such as ISO594/ISO80369 that relate to small-bore connections for direct connection and use with the body such as syringes, fluid and drug introduction accessories and catheters. These standards are specifically orientated towards ensuring compatibility and to ensure fluid connections are robust and to avoid leaks for devices that are intended to be connected together rather than to specifically avoid connectivity where it is not intended. These standards also do not provide for monitoring of the connection. These standards do not directly relate to the connector applications and devices as described in the present invention which combines differing aspects such as physical insertion, connectivity and the enabling of function. The connector applications according to the present invention may generally but not exclusively relate to larger bore connectors.

The present invention seeks to mitigate potential hazards due to misconnection by only allowing the intended devices to operate and hence maintain patient safety and also provide the opportunity for warning alarms to alert clinicians if incompatible devices are sensed as being connected to together.

The present invention described herein goes beyond the prior art in this field through the addition of the sensing and monitoring of connector insertion to detect the degree of insertion.

Even within the limited scope of compression garments intended for VTE prophylaxis, there are many different devices that are not intended to be connected together but which conceivably could physically be mated if either the instructions were not followed or alternatively if they were fitted together in error by a user. Further to this aspect, even compatible and approved constituent parts or accessories that are intended to be physically connected together can suffer a lack of function if the connection is not made correctly and completely by the user. Sometimes the user unknowingly only partially inserts the connector and leaves it in a state that is not fully attached. This can result in delays to treatment and complications within a clinical setting such as leaks or alarm conditions.

The present invention therefore covers the combination of approved accessories so that the full connection is identified and correct operation is therefore ensured. This connection status can then be monitored repeatedly during clinical use.

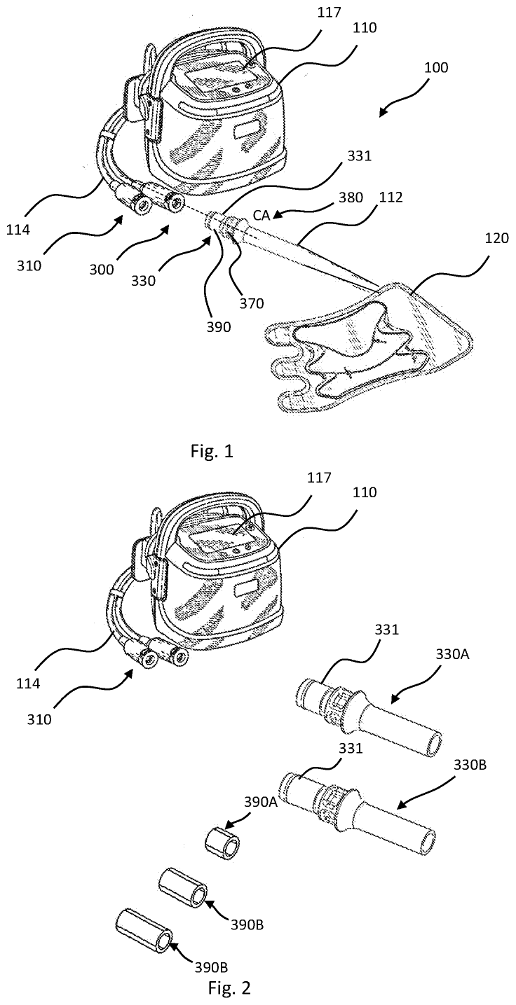

FIG. 1 depicts a fluid pressure control system according to an embodiment. The fluid pressure control system 100 may be a gas pressure control system such as a pneumatic control system or may be based on any type of suitable fluid for the application with inflatable/deflatable articles.

The fluid pressure control system 100 comprises an inflatable/deflatable article 120, a pump 110 and a controller (not shown in FIG. 1) operatively connected to the pump 110. The controller is operatively connected to the pump 110 for controlling said pump 110.

The pump 110 may be a pneumatic pump. The pump 110 may be arranged to control fluid flow to and from the inflatable/deflatable article 120. Accordingly, the pump 110 may be arranged to inflate or deflate the inflatable/deflatable article 120.

The fluid pressure control system 100 further comprises a coupling assembly 300 for fluidically connecting the inflatable/deflatable article 120 and the pump 110. The coupling assembly 300 comprises a connector 330 and a connecting member 310.

The connector 330 is connectable to the connecting member 310 to upon engagement form a fluid pathway through said connector 330 and the connecting member 310. Accordingly, the connector 330 may be connectable to the connecting member 310 to allow for fluid communication through the coupling assembly 300.

The coupling assembly comprises a position indicating arrangement 380. The position indicating arrangement 380 comprises a position indicating part 390 provided on the connector 330 or the connecting member 310 and a sensing arrangement (not shown in FIG. 1) for detecting the position of the position indicating part 390 relative said sensing arrangement during engagement between said connector 330 and connecting member 310.

Thus, a partial engagement between the connecting member and the connector may be detectable by means of the monitoring of the position of the position indicating part. Accordingly, the risk for misconnection is reduced.

Engagement between the connector 330 and the connecting member 310 herein refers to a state wherein the connector 330 and the connecting member 310 are in contact. In said state, the connector and the connecting member may be connected, non-connected or partially connected.

In one embodiment, the position indicating part 390 is provided on the connector or the connecting member 310 and the sensing arrangement is at least partly provided on the other of the connector 330 or the position indicating part 390.

Accordingly, at least some of the components of the sensing arrangement may provided on the other of the connector 330 or the connecting member 310.

In an alternative embodiment, the sensing arrangement may be provided in a separate component.

In one embodiment, the sensing arrangement is provided on the other of the connector 330 or the connecting member 310.

In one embodiment, the connector 330 comprises a distal part 331. The distal part 331 extends in a distal direction of the connector 330.

The connector 330 and the connecting member 310 are connectable to form the fluid pathway through the connector 330 and the connecting member 310 by means of insertion of the distal part 331 of the connector 330 into the connecting member 310.

The distal part 331 is movable inside the connecting member 310 along a connection axis CA. The connection axis CA extends distally from the connector 330, The distal part 331 is movable inside the connecting member 310 along the connection axis CA between a coupled position and a non-coupled position. Preferably, the distal part 331 and the connecting member 310 are adapted to sealingly engage when the distal part 331 is in the coupled position.

The distal part 331 may be movable from a non-inserted position to the non-coupled position. In the non-coupled position, the distal part 331 may have at least come into contact with the connecting member 310. A non-coupled position herein refers to a position of the distal part 331 inside the connecting member 310 wherein the coupling assembly does not provide an intended fluid communication through the connecting member 310 and the connector 330. Correspondingly, a coupled position herein refers to a position of the distal part 331 inside the connecting member 310 wherein an intended fluid communication through the connecting member 310 and the connector 330 is achieved.

Substantially the entire length of the distal part 331 may be inserted into the connecting member 310 when the distal part 331 is in the coupled position.

Further, a distal end of the distal part 331 may be adjacent to a proximal end of the connecting member 310 when the distal part 331 is in the non-coupled portion. In one embodiment, the distal end of the distal part 331 is positioned inside the connecting member 310 when the distal part 331 is the non-coupled position.

The coupling assembly 330 further comprises a position indicating arrangement 380. The position indicating arrangement 380 is configured to generate a position signal to the controller. The position signal is indicative of the position of the distal part 331 relative to the connecting member 310. Thus, the position signal may be indicative of the position of the distal part 331 relative to the connecting member 310 along the connection axis CA.

The position indicating arrangement 380 comprises a position indicating part 390. The position indicating part 380 is provided on the connector 330 or the connecting member 310. The position indicating arrangement 380 further comprises a sensing arrangement (not shown in FIG. 1). The sensing arrangement is for detecting the position of the position indicating part 390 when the distal part 331 is inserted into the connecting member 310.

The position signal is based on the position of the position indicating part 390 provided on the connector 330 or the connecting member 310 relative the other of the connector 330 or the connecting member 310.

The sensing arrangement enables fast feedback to the user, which results in the sensing being responsive (for example <2 second response time) and so reduces the need for the clinician to wait for an inflation to ensure the physical connection is correct and the pump can correctly operate with the coupled inflatable/deflatable article. Further, the inflatable/deflatable article can also be sensed when being disconnected immediately rather than waiting for up to 1 minute for the inflation cycle to occur and sense the resulting lack of inflation due to the lack of a connected article.

Hence, the present invention includes an insertion monitoring system that can differentiate between a partial connection situation where the position of connector is such that any mechanical latch mechanism located on connecting member 310 and/or connector 330 will not be fully engaged and a full insertion connection situation where the mechanical latch will be engaged. This differentiation is performed in a non-contact manner and it is provided independent of the latch mechanism itself as it is based on sensing relative connector position.

This can be achieved through the real-time monitoring of the position of the position indicating part. Hence it represents an improvement and extension to the coupling assemblies found in the prior art.

In one embodiment, the position signal may be associated with a corresponding degree of insertion of the distal part 331 into the connecting member 310.

In one embodiment, the pressure provided by the pump 110 is controlled based on the position signal such that the pressure provided by the pump 110 is dependent on the position of the connector 330, 630 relative to the connecting member.

The coupling assembly according to the invention can be used with fluid-based inflatable/deflatable garments and their associated pumps or alternatively more generally with any type of compression system used on the limb of a patient involving a two-part connection of controller and limb compression device. A number of alternative compression systems and technologies exist where the controller is separable from the patient-fitted compression device. Some compression systems use air connections but others use electrical or other types of physical connections. The present invention allows for the coupling assembly to be monitored independent of the flow/control mode of operation. This offers flexibility and hence can be used with a variety of connections between separate connectable parts of a patient compression system. Thus, a more flexible coupling assembly and fluid pressure control system is achieved.

There are also wider applications of the invention as it can be used outside of compression systems in other medical areas involving the connection of any two-part system. Examples of application are as detailed in U.S. Pat. No. 6,884,255 and include the connection of pumps to support surfaces such as mattresses and/or seatpads for the prevention and/or treatment of pressure related injuries (such as decubitus ulcers/pressure injuries), Further examples include connections between pumps and garments for the treatment of circulatory conditions such as lymphedema as well as other two-part medical products such as blood pressure monitors. It will be obvious to any reader skilled in the art that other medical applications exist involving the connection of a variety of patient-worn devices to separate control units exist for treatment and/or monitoring purposes and therefore these are also intended to be included within the scope of the invention.

According to an embodiment, the inflatable/deflatable article is a medical inflatable/deflatable article such as a garment. The term garment is generically used and intended to cover and apply to both traditional inflatable compression garments and also any other limb-fitted compression device or medical device intended for use with a patient or human being and remaining in place on part of the body. Referencing FIG. 1, the garment 120 may be a compression sleeve.

As is known to the skilled person, the pump 110 may have a rotor and stator or valve unit to control the fluid flow to inflate or deflate the inflatable/deflatable article.

As depicted in FIG. 1, the connector 330 is configured to be fluidically connected to the inflatable/deflatable article 120. Thus, the connector 330 may be associated with an inflatable/deflatable article 120. The connecting member 310 is configured to be fluidically connected to the pump 110. Thus, the connecting member 310 may be associated with a pump 110.

The connector 330 may be fluidically connected to the inflatable/deflatable article 120 by means of an article fluid connection 112. The article fluid connection 112 may be a tube or a hose.

The connecting member 310 may be fluidically connected to the inflatable/deflatable article by means of a pump fluid connection 114. The pump fluid connection 114 may be a tube or a hose.

According to an embodiment, the coupling assembly 300 may further comprise a mechanical latch 370. The mechanical latch 370 is arranged to secure the distal part 331 in the coupled position. The coupled position may thus be a latched position of the distal part.

The mechanical latch 370 may be a manually operated mechanical latch adapted to be engaged by a user to secure the distal part 321 when said distal part 321 is in the coupled position.

Alternatively, the mechanical latch 370 is adapted to resiliently engage to secure the distal part 331 when said distal part 331 is in the coupled position.

Preferably, the mechanical latch 370 comprises a locking member provided on the connecting member 310 or the connector 330 and a retention member provided on the other of the connecting member 310 or the connector 330. When the distal part 331 is in the coupled position, the retention member is arranged to engage the retention member, whereby the mechanical latch 370 is secured relative to the connecting member 310.

Mechanical latches are well-known in the prior art and will not be described in further detail.

Preferably, the position indicating part 390 is provided in a proximal position of the connector 330 or the connecting member 310 such that said position indicating part 390 moves along the connection axis CA when the distal part 331 moves distally between the non-coupled position and the coupled position.

Thus, the position signal is generated throughout the entire movement of the distal part inside the connecting member. Thereby, an exact indication on the degree of insertion of the distal part is provided. Thus, the position indicating part may move a longer distance along the sensing arrangement (sensor unit) during coupling which allows for more exact determination of the position of the position indicating part.

Further referencing FIG. 1, the fluid pressure control system 100 further comprises an indicating device 117. The indicating device 117 is operatively connected to the controller. The indicating device 117 is configured to provide an indication to a user based on the position signal.

Accordingly, the indicating device 117 may be configured to provide an indication to a user in response to distal part 331 being in the coupled position and/or the non-coupled position.

The indicating device 117 may be provided on the pump 110. Accordingly, the indicating device 117 may be mounted to the casing of the pump 110. In one embodiment, the indicating device can also be mounted on the connecting member 310. Thus, the user may be provided an indication while operating the pump 110.

In one embodiment, the indicating device 117 may be a display unit, such as an LCD-display.

The indicating device 117 may be configured to provide an indication to a user in response to the distal part 331 being in an intermediate position along the connection axis CA between the coupled position and the non-coupled position.

In one embodiment, the controller is provided on or within the pump 110.

In one embodiment, the controller may comprise a position indication timer. The indicating device 117 may be configured to provide an indication to a user within a time interval in response to the distal part 331 being moved from the non-inserted position to the non-coupled position. Preferably, the time interval is 5 seconds. Accordingly, the indicating device 117 may be configured to provide an indication to a user within 5 seconds of the insertion of the distal part 331.

Turning to FIG. 2 different types of position indicating parts 390A, 390B, 390C in conjunction with a fluid pressure control system are depicted.

According to an embodiment, the position indicating part 390 may be provided on the distal part 331 of the connector 330. Preferably, the position indicating part 390 may have a length extending along the connection axis CA of more than 2 mm. In order to allow for differentiating between different types of components of the fluid pressure control system, the size, material characteristics and shape of the position indicating part 390 may vary,

The position indicating part 390 may have a generally cylindrical or toroidal shape. Advantageously, the position indicating part 390 has an outer dimension (i.e. maximum width or height orthogonal to the connection axis) of between 5 and 10 mm and preferably between 6 and 8 mm. The position indicating part 390 may have an inner dimension (i.e. an inner diameter) of preferably less than 6 mm and more preferably greater than 4 mm. Thus, the position indicating part 390 may be annular. This may allow for sufficient fluid flow through the position indicating part.

The position indicating part 390 may have a length extending along the connection axis of between 1 and 10 mm and more preferably between 2 and 9 mm.

A number of further alternative embodiments of position indicating parts 390 exist that are within the scope of the invention and should be obvious to anyone skilled in the art of position sensing and object detection.

According to an embodiment, the coupling assembly 300 may further comprise a component identifying means. The component identifying means may be provided on the connector 330 or the connecting member 310. The component identifying means may serve to identify the inflatable/deflatable article (not shown in FIG. 2) or pump 110.

Thus, the coupling assembly and fluid pressure control system may provide both an identification feature and an insertion monitoring feature which further reduces the risk for misconnections and partial connections.

Advantageously, the component identifying means are integrated into the position indicating part 390. Thus, the position indicating part 390 may be configured so as to be associated with the inflatable/deflatable article or pump 110, i.e. serve to identify the inflatable/deflatable article or the pump 110.

The component identifying means may be configured to generate a characteristic response while the distal part 331 is inserted into the connecting member 310. Said characteristic response may be detectable by means of the sensing arrangement. Alternatively, said characteristic response may be detectable by means of a separate component sensor. Such a component sensor may be operatively connected to the controller.

The controller may be configured to compare the characteristic response with a set of stored characteristic responses. The set of stored characteristic responses are associated with a corresponding set of pumps 110 or inflatable/deflatable articles to identify and confirm the compatibility of pump or inflatable/deflatable article.

The set of stored characteristic responses may be stored in a memory of the controller.

In one embodiment, the controller is configured to selectively deactivate the position indicating arrangement in response to the comparison with the set of stored characteristic responses. In a further embodiment, the controller is configured to activate or deactivate the position indicating arrangement in response to the comparison with the set of stored characteristic responses. Accordingly, the controller may be configured to enable or disable the position indicating arrangement in response to the comparison with the set of stored characteristic responses.

In one embodiment, the position indicating part 390 is in the form of identification component. The position indicating part may be adapted to generate the characteristic response associated with the inflatable/deflatable article 120 or the pump 110.

In one embodiment, the characteristic response may be detectable by means of the sensing arrangement for identifying the inflatable/deflatable article 120 or the pump 110.

In one embodiment, the characteristic response may be detectable when the distal part is in the coupled position, i.e. the characteristic response is generated when the distal part is in the coupled position and the identification component is in a corresponding position relative the sensing arrangement, e.g. a sensor unit of the sensing arrangement.

The combination of the position indicating arrangement and the component identifying means allows for the pump to only be operated when the coupling assembly is properly connected and the connected article is deemed compatible, whereby a safer and more reliable fluid pressure control system is achieved.

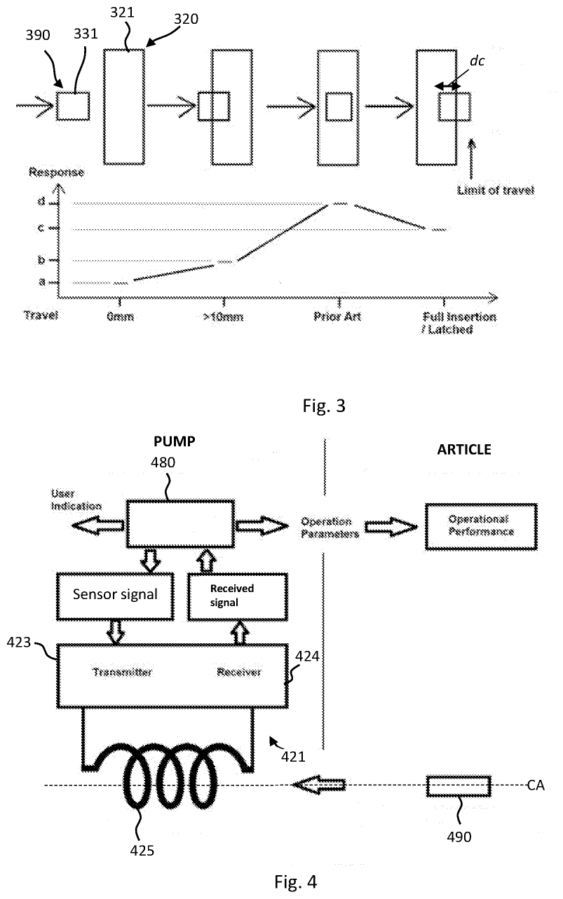

FIG. 3 depicts a schematic diagram of the operation of a fluid pressure control system and the insertion of the distal part 331 into the connecting member 330.

The position signal may be based on a measured value obtained by the sensing arrangement 320. Accordingly, the sensing arrangement 320 may be configured to obtain a measured value based on the position of the position indicating part 390 provided on the connector or the connecting member relative the other of the connector or the connecting member.

The measured value may be associated with a detected transient maximum when the distal part is between the non-coupled position and the coupled position and a detected lower steady state when the distal part is in the coupled position.

In one embodiment, the detected transient maximum has a measured value (as shown graphically as value ‘d’ in FIG. 3) that is >10% larger than the detected lower steady state in the coupled position (as shown as value ‘c’ in FIG. 3).

The monitoring of the coupling of the coupling assembly involves the following elements in the form of sequentially functional steps.

Initially, the distal part 331 is aligned with the connecting member. The connector is aligned along the connection axis CA. Thereby, the distal part 331 can be inserted into the connecting member.

Preferably, the distal part 331 is not detectable by the sensing arrangement 320 until said distal part 331 has travelled a distance into the connecting member. This shown as the response of value “a” in FIG. 3. Accordingly, the distal part 331 is partly inserted into the connecting member 310 in the non-coupled position of the distal part 331 detectable by the sensing arrangement.

Further referencing FIG. 3, an increasing insertion of the distal part 331 causes an increase measured value obtained by the sensing arrangement 320. Accordingly, a movement of the distal part 331 from the non-coupled position to the coupled position causes an increase in the measured value obtained by the sensing arrangement 320.

The measured value increases until the distal part 331 reaches a position associated with a maximum measured value, i.e. the detected transient maximum, as shown with values “b”−“d”. Said position is between the coupled position and the non-coupled position.

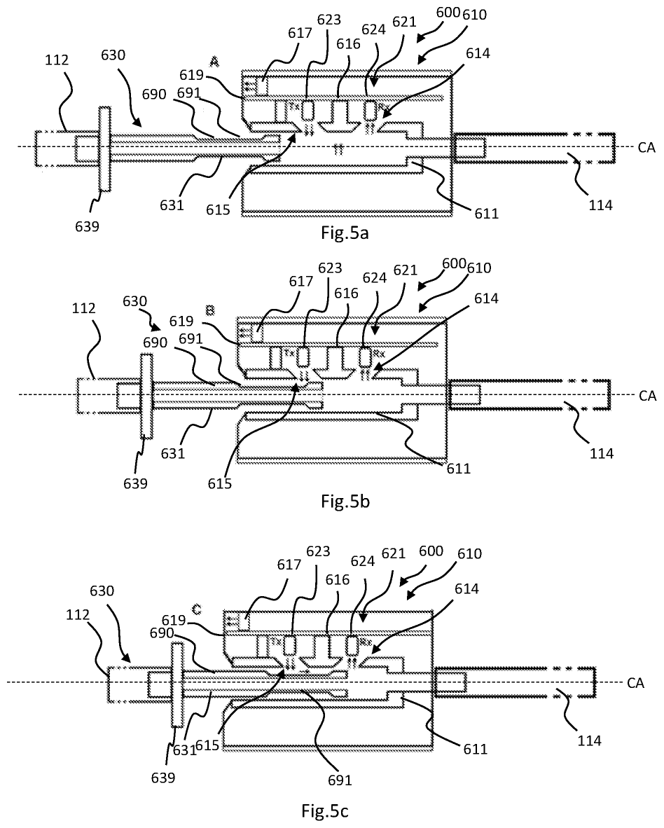

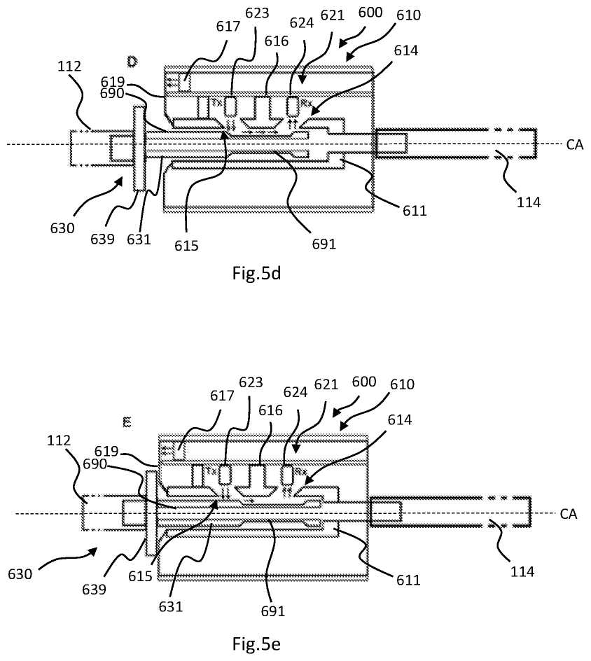

Further insertion of the distal part 331 beyond the aforementioned position results in the measured value decreasing, as shown with the value “c”. The coupled position is reached beyond the aforementioned position. As will be described in further detail with reference to FIG. 5a-e, the coupling assembly may comprise a limit stop to stop insertion beyond the coupled position.

When the distal part 331 reaches the coupling position an indication may be provided to the user by means of the indicating device.

In one embodiment, the travel distance for the distal part 331 between a first non-inserted position with no response to a second position with a response is at least 10 mm.

In another embodiment, the travel distance for the distal part 331 between a first position with a detectable response to a second position where the distal part is coupled is at least 10 mm.

In another embodiment, the travel distance for the distal part 331 between a first uncoupled position with a maximum response signal and a second coupled position with a reduced response signal is at least 2 mm.

Further, the monitoring of the de-coupling of the coupling assembly involves the following elements in the form of sequentially functional steps. Monitoring of the de-coupling of the coupling assembly prevents the connector being retained in an intermediate position during de-coupling of the coupling assembly.

In the coupled position, the sensing arrangement 320 continuously obtain a measured value under a steady state, as shown with the value “c”.

The user may then disengage the mechanical latch to allow for movement of the distal part 331.

The measured value obtained by the sensing arrangement 320 then gradually increases when the distal part 331 is moved from the coupled position towards the non-coupled position until the distal part 331 reaches the position associated with the maximum measured value as shown with value “d”, i.e. the detected maximum response.

When the measured value obtained by the sensing arrangement exceeds a threshold indicative of the coupled position stored in the controller, i.e. the memory associated with the controller, the position signal indicates that the distal part 331 no longer is in the coupled position. This exemplified by the value “c” in FIG. 3.

When the position signal indicates that the distal part 331 no longer is indicative of the distal part 331 being in the coupled position an indication may be provided to the user by means of the indicating device.

Further movement of the distal part 331 beyond the aforementioned position results in a decrease in the measured value obtained by the sensing arrangement 320 as shown by “b”.

In one embodiment, the component identifying means are arranged to generate a response detectable by the sensing arrangement 320 when the distal part 331 is in the non-coupled position to identify the pump or inflatable/deflatable article.

In an embodiment during decoupling, the travel distance between a first coupled position with a non-maximum response signal and a second uncoupled position associated with a maximum response signal is at least 2 mm.

Additional movement of the distal part 331 to a non-inserted position beyond the non-coupled position causes a decrease in the measured value by the sensing arrangement 320 to the measured value which is the same as before insertion of the connecting member 310, shown by “a”.

In a further embodiment, the travel distance between a first uncoupled position with a maximum response signal and a second uncoupled position with no response signal is at least 10 mm.

Upon the distal part 331 reaching the non-inserted position an indication may be provided to the user by means of the indicating device.

In the embodiment depicted in FIG. 3, the position indicating part 390 is provided on the connector and more specifically the distal part 331. The sensing arrangement 320 comprises at least one sensor unit 321. As will be described with reference to FIGS. 4 and 5 the sensing arrangement may utilise different types of sensing technology.

In one embodiment, a central point of the position indicating part 390 is arranged to be at a distance dc along the connection axis CA in a distal direction from a center point of the sensor unit 321 of the sensing arrangement 320 when the distal part 331 is in the coupled position. Further, the sensor unit 321 may be provided on the connecting member. The distance dc may be a distance relative a center point of the sensor unit, i.e. an offset distance from said center point.

In one embodiment, the distance dc may be between 1 mm and 0.01 mm and preferably less than 0.5 mm. In one embodiment, wherein the sensor unit 321 comprises a coil (as will be described in further detail with reference to FIG. 4), the distance dc may be a distance from the center point of said coil. Thus, the coil may overlap the position indicating part. The aforementioned distance allows for accurate sensing of the position of the position indicating part.

FIGS. 4 and 5 depicts aspects of a coupling assembly and fluid pressure control system according to two alternative embodiments, i.e. a coupling assembly and fluid pressure control system implementing radio-based sensing and a coupling assembly and fluid pressure control system implementing optical sensing.

The sensing arrangement 320 may be a non-contact type of sensing arrangement.

The use of a non-contact based sensing is particularly advantageous as it avoids a number of issues associated with potential alternative embodiments that use a physical contact means such as problems associated with the buildup of debris/material on contacts, regulatory concerns regarding exposed electrical contacts and physical damage to the alignment of a contact.

Referencing both FIGS. 4 and 5, the sensing arrangement 320 may comprise a transmitter 423, 623 and a receiver 424, 624. The transmitter 423, 623 is configured to generate a sensor signal. The sensor signal is received by the receiver 424, 624. The position signal is based on the characteristics of the signal received by said receiver 424, 624. The signal received by the receiver 424, 624 may be considered the measured value obtained by the sensing arrangement.

Accordingly, the transmitter 423, 623 is configured to generate a sensor field along the connection axis CA. The position signal may be generated based on the position of the position indicating part 490, 690 obtained by the receiver 424, 624.

With reference to FIG. 4, the sensing arrangement may be an induction based sensing arrangement. Thus, the sensor device may be an induction sensor. Preferably, the sensing arrangement may be a radio-based sensing arrangement. With reference to FIG. 5a-5e, the sensing arrangement may be an optical based sensing arrangement utilising a variable degree of transmittance of light or infrared based on the degree of coupling engagement shown in FIGS. 5a to 5e. Thus the sensor device may include a Light Emitting Diode (LED) for transmitting and an optically-receptive sensor such as a photodiode for receiving.

Further referencing FIG. 4, the sensor unit 421 may further comprise a sensor coil 425. The sensor coil 425 may be operatively connected to the transmitter 423 and the receiver 424. In one embodiment, the sensor coil 425 may be arranged to be coaxial to the connection axis CA.

The sensor coil 425 may be configured to electromagnetically couple the transmitter 423 and the receiver 424.

Said sensor coil may be configured to generate an electromagnetic field extending along the connection axis CA, whereby the position indicating part 490 is detectable inside said electromagnetic field. The position indicating part 490 causes a change in the received signal compared to the sensor signal indicative of the position and/or movement of the position indicating part inside said electromagnetic field.

Accordingly, the length, configuration and/or dimensions of the sensor coil 425 may be chosen such that the electromagnetic field extends along said connection axis CA.

The position indicating part 490 may preferably be made of a material selected from a group consisting of a ferrite material, steel and a brass material. The position indicating part 490 may be fitted to the distal part of the connector. For example, the position indicating part may be a ferrite ring, in a toroidal format. Other materials can provide a similar effect such as certain grades of steel and brass.

The material (for example ferrite) in the position indicating part 490 forms a variable permeability core to the sensor coil 425. Thereby, the coil inductance is modified. This change in inductance can be detected by means of electrical circuitry in the controller 480 as a phase change in the sensor coil current resulting from the applied waveform signal and also as an amplitude change to the current flowing in the sensor coil 425. This change is a time-varying function of the degree of insertion of the distal part of the connector and the identification device into the sensor coil 425. According to the embodiment depicted in FIG. 4, the sensor coil 425 may be provided on the connecting member which may be associated with the pump.

In one embodiment, the sensor unit may be formed by the sensor coil 425 (with the receiver and transmitter). The sensor coil 425 may have a length along the connection axis of between 2 mm and 10 mm and preferably approximately 5 mm. The position indicating part may be between 1 mm and 5 mm, preferably between 3 and 4 mm so the central point of the position indicating part can be offset from the central point of the sensor coil.

The controller 480 may be configured to continually monitor the position signal and compare the measured value with reference measured values stored in the memory of said controller 480.

Changes between the sensor signal and the received signal are indicative of the position or movement of the position indicating part 490. Such changes may be in the form a changed amplitude and phase in the received compared to the sensor signal. The received signal may be configured to be processed by the controller 480. Said controller 480 may be further configured to measure the phase change between the sensor signal and the received signal. The frequency of the received signal compared to the sensor signal may be largely unchanged.

As aforementioned, the sensing arrangement may utilize an inductive sensing method involving a radio frequency based measurement. Preferably, the sensing arrangement may operate on frequencies of at least 80 kHz, less than 300 kHz and preferably around 125 kHz.

In one embodiment, a single coil may be used to transfer the sensor signal between the transmitter and receiver. Accordingly, the transmitter 423 and receiver 424 may be in electrical connection with the sensor coil 425. In one embodiment, the sensor coil 425 may be mounted in the connecting member. In one embodiment, may be mounted in the casing of the pump.

Other embodiments within the scope of the invention include the use of a split coil with independent connections/windings where the transmit and receive signals are separate. Thus the sensor unit 421 may comprise a receiver coil and a transmitter coil, whereby the received signal is separate from the sensor signal, i.e. the signal transmitted from the transmitter coil.

Alternatively, it is also possible to use separate transmit and receive coil arrangements where the two coils are always used for different purposes.

In one embodiment, the sensor coil 425 is arranged to allow for fluid flow through a central axis of said sensor coil 425. The central axis of the sensor coil 425 may be substantially aligned with the connection axis CA.

In one embodiment, the sensor coil 425 may be in the form of a ‘Brook coil, i.e. it being dimensioned according to the well established ‘Brooks coil’ relative dimensions to allow for manufacturing efficiencies in coil winding and maximizing the resulting inductance provided by the wire used in the coil. This dimensional requirement is extended such that the sensor coil 425 may have a length of 5 mm in the direction of the connection axis CA. This allows ensures that the majority of the resulting electromagnetic field can be entered and exited during the insertion process, Hence the transition of the position indicating part 490 can provide the necessary response signals upon entering and transition through the coil as shown in FIGS. 3 and 4.

The aforementioned coil dimensioning helps to optimize operation in use, improve coupling and reduces the physical size requirements whilst ensuring maximum coil sensitivity to the introduced position indicating part material.

This optimal dimensioning involves ensuring the ratio of the inner diameter of the sensor coil 425, (which may form the path for the distal part of the connector and therefore the fluid flow), to the coil length being at least 2 and preferably the ratio of sensor coil 425 outer diameter to position indicating part length being at least 5.

In one embodiment, the sensor coil 425 has an inductance of 400-500 uH, preferably 446 uH when no position indicating part 490 is present in the coil. The inductance changing in response to the presence of the position indicating part 490.

In one embodiment, the system as whole (formed from coil, associated pump circuitry and position indicating part) is tuned to be resonant between 80 kHz and 300 kHz and preferably around 125 kHz. Accordingly, the pump, the sensor unit 421 and the position indicating part 490 may be tuned to operate between 80 kHz and 300 kHz and preferably around 125 kHz.

The transmitter 423 and the receiver 424 may be provided on the connector or the connecting member or as an alternative, externally from said connector or connecting member such as on the body (casing) of the pump.

In one embodiment, the transmitter 423 and the receiver 424 may be provided on the connecting member or the connector. The transmitter 423 and the receiver 424 may be provided on the connecting member or the connector together with the sensor coil 425. Thus, the transmitter 423 and the receiver 424 may be comprised in the sensor unit 421.

In one embodiment, the transmitter 423 and the receiver 424 are arranged externally from the connecting member and/or the connector. For example, said transmitter 423 and receiver 424 may be arranged on the pump. Thereby, the electronics of the system may be kept together on a single PCB which is advantageous both from a cost and complexity standpoint. Further, this allows for a connecting member or connector without costly electronic components which makes it easier and cheaper to replace.

Referencing FIG. 5a-e, an embodiment which involves either alternatively or additionally using transmitted light within the connector and the connecting member is depicted. According to this embodiment, the reflective response provides the same type of change in the received signal compared to the sensor signal as detailed above with reference to FIGS. 3 and 4.

Accordingly, the sensing arrangement may be an optical sensing arrangement. The sensor unit 621 may thus be an optical sensing unit.

The transmitter 623 and the receiver 624 may be comprised in the sensor unit 621.

The transmitter 623 may be configured to transmit a sensor signal in the form of an optical signal to the receiver 624. The position signal is thus based on the received signal, i.e. received optical signal, in the receiver 624. The optical signal can be in the visible spectrum or can involve the use of Infra Red (IR) illumination. Accordingly, the optical sensor unit may be an IR-sensing unit or a visible light sensor unit, such as a photodiode-based sensor unit.

As described with reference to FIG. 3, the controller may be configured to compare the measured value obtained by the sensing arrangement, i.e. the sensor unit 621, in the form an amplitude of the received signal in the receiver 624, with predefined values stored in the controller. Thus, the position of positon indicating part may be obtained. The process described with reference to FIGS. 3 and 4 thus applies analogously to the embodiment depicted in FIG. 5a-e.

Further referencing FIG. 5a-e, the position indicating part 690 may together with an outer surface of the distal part 631 facing the position indicating part 690 be adapted to provide an optical pathway for the sensor signal to the receiver 624. Alternatively, the position indicating part 690 may together with an inner surface of the connecting member 610 facing the position indicating part 690 be adapted to provide an optical pathway for the sensor signal to the receiver 624. Said optical pathway may extend between the transmitter 623 and the receiver 624.

The optical pathway may be alterable based on the position of the position indicating part 690 between the receiver 624 and the transmitter 623, thereby causing a variation of the signal received by the receiver 624 indicative of the position of the position indicating part 690. Thus, the optical pathway may change based on said position of the position indicating part 690.

It is noted that the optically based position indicating part can be achieved by the use of different sized, textured and colored position indicating parts fitted to the distal part 631 or the connecting member 610 and the selection of material for said distal part 631 or connecting member 610. The details of the position indicating part can affect the degree of alignment of the light transmitted from the transmitter 623 to the receiver 624 within the connecting member 610.

The resulting light transmission level, i.e. the sensor signal received by the receiver 624, i.e. the measured value, can therefore be modified as the distal part 631 is positioned between the coupled position and the non-coupled position. This can be used to provide the same signal benefits of the embodiment as shown in FIG. 3.

The position indicating part may comprise any one of the following: a reflective outer material, an uneven contour or texture, polished surface or an indicating color. These features provide means for modifying the received optical level based during the detection of relative position of the position indicating part

According to the embodiment depicted in FIG. 5a-e, the position indicating part 690 is provided on the distal part 631. The position indicating part 690 and the inner surface of the connecting member 610 are adapted to provide the optical pathway for the sensor signal to the receiver 624 (and from the transmitter 623).

The connecting member 610 may comprise a connecting barrel 611. The connecting barrel 611 is adapted receive the distal part 631. The inner surface of the connecting barrel 631 faces the position indicating part 690 when the distal part 631 is in the non-coupled positon, the coupled position and between the non-coupled and coupled position.

The sensor unit 621 is provided on the connecting member 610. The sensor unit 621 may be mounted to the connecting member 610 by means of a mounting structure 619.

The inner surface of the connecting member 610, i.e. the connecting barrel 611, may comprise a first aperture 615. The first aperture 615 is arranged to allow passage of the sensor signal transmitted from the transmitter 623 into the connecting barrel 611. The inner surface of the connecting member 610, i.e. the connecting barrel 611, may comprise a second aperture 614. The second aperture 614 is arranged to allow passage of the sensor signal transmitted from the transmitter 623 from the connecting barrel 611 to the receiver 624 and hence to the controller.

The inner surface of the connecting member 610, i.e. the connecting barrel 611, may further comprise a separating part 616. The separating part extends between the first aperture 615 and the second aperture 614. The separating part 616 may extend along the connection axis CA.

In one embodiment, the position indicating part 690 comprises a depression 691. The depression 691 is arranged to together with the separating part 616 provide an optical pathway for the sensor signal transmitted from the transmitter 623. The depression 691 may be arranged to be radial relative to the connection axis CA.

The processing of the signal received by the receiver in the embodiment in FIG. 5a-e is analog to the embodiment described in FIGS. 3 & 4. Thus, the variation in the signal received can be detected by means of electrical circuitry in the controller. This change is a time-varying function of the degree of insertion of the distal part of the connector and the identification device into the connecting member.

The controller is thus configured to continually monitor the position signal and compare the measured value with reference measured values stored in the memory of said controller.

Changes between the sensor signal and the received signal are indicative of the position or movement of the position indicating part 690. Said changes in the sensor signal may be obtained by the controller in the form of changes in a measured voltage from the receiver 624.

Further referencing FIG. 5a-e, the indicating device 617 may be a light source. Said light source may be operatively connected to the controller for providing the user with an indication based on the position signal. Said indication may be in the form of the color of the light changing, the light flashing etc.

In one embodiment, the indicating device is in the form of lighting device. The lighting device may be mounted to the connecting member 630.

In one embodiment, the sensor device 621 is a visible light sensor device. In such an embodiment, the light emitted from the transmitter 623 may constitute the light source adapted to provide a user an indication based on the position signal.

Preferably, the light source is proximal to the connector 630 when the distal part 631 is in the coupled position.

FIG. 5a-e further depicts a coupling sequence in the steps A-E. Although here depicted for an embodiment based on optical sensing, it may also be equivalent and applicable to the conduction sensing based embodiment depicted in FIGS. 3 & 4.

In step A, the distal part 631 is just inserted into the connecting member 610. The distal part 631 is thus in the non-coupled position. The position indicating part 690 is not positioned between the transmitter 623 and the receiver 624. Thus, the sensor signal received by the receiver 624 indicates that no position indicating part is present. In this example, there is only a minor optical coupling between the transmitter 623 and receiver 624 to indicate that said position indicating part 690 is not present.

In step B, the distal part 631 is further inserted into the connecting member 610. The distal part 631 is thus in a first intermediate position between the non-coupled position and the coupled position. The position indicating part 690 is inserted to a position between the transmitter 623 and the receiver 624. Thus, the position indicating part 690 sufficiently interferes with the sensor signal to be detectable by the sensor device 621. In this example, the position indicating part 690 sufficiently obscures the light signal from the transmitter 623 from reaching the receiver 624. This results in a reduced degree of optical coupling relative the start of the insertion, i.e. the non-coupled position.

In step C, the distal part 631 is further inserted into the connecting member 610 compared to step B. The distal part 631 is thus in a second intermediate position between the non-coupled position and the coupled position. The position indicating part is also largely positioned between the transmitter 623 and the receiver 624, whereby interference of the sensor signal is changed compared to step B. In this example, the depression 691 of the position indicating part 690 starts to provide an increase in the optical coupling between the transmitter 623 and the receiver 624.

In step D, the distal part 631 is further inserted into the connecting member 610 compared to step C. The distal part 631 is thus in a third intermediate position between the non-coupled position and the coupled position. Compared to step C, the position indicating part 690 is to a larger extent positioned between the transmitter 623 and the receiver 624, i.e. a larger portion of the position indicating part 690 is positioned between said transmitter and receiver than in step C. The position signal thus reaches a maximum transient value. In this example, the position indicating part 690 provides a maximum degree of optical coupling between the transmitter 623 and the receiver 624.

In step E, the distal part 631 is fully inserted into the connecting member 610. The distal part 631 is thus in the coupled position. Compared to step D, the position indicating part 690 is to a lesser extent positioned between the transmitter 623 and the receiver 624, i.e. a smaller portion of the position indicating part 690 is positioned between the transmitter 623 and the receiver 624 than in step D. In this example, the degree of optical coupling between the transmitter 623 and the receiver 624 is reduced to a level below step D.

As depicted in FIG. 5a-e, the coupling assembly may comprise the limit stop 639. The limit stop 639 may be provided in a proximal position of the distal part 631. The limit stop 639 is arranged to come into contact with the proximal end of the connecting member 610 when the distal part 631 is in the coupled position, thereby providing a mechanical stop for the insertion of the distal part 631.

The limit stop 639 may in one embodiment be in the form of a collar extending radially from the distal part 631. In one embodiment, the limit stop may be in the form of a protrusion or a control pin.

According to an aspect a pump system configured to be connected to an inflatable/deflatable article 120 in a fluid pressure control system 100 is provided. The pump system is configured to be connected to the inflatable/deflatable article 120 by means of a coupling assembly according to any one of the previously described embodiments.

The pump system comprises the pump 110 and a controller 480 operatively connected to the pump 110 for controlling said pump 110 and the sensing arrangement 320.

The pump system may be configured to be connected to the inflatable/deflatable article 120 by means of a coupling assembly. The pump system further comprises an indicating device 117, 617 operatively connected to the controller 480. The indicating device 117, 617 is configured to provide an indication to a user based on the position signal obtained by the controller 480 from the position indicating arrangement 380.

The indicating device 117, 617 may be provided on the pump 120.

The indicating device 117, 617 may be configured to provide an indication to the user at a position proximal to the connector 330, 630.

The controller 480 may be configured to control the pump 110 based on the position signal obtained by the controller 480 from the position indicating arrangement 380.

The controller may be configured to control the pump based on the characteristic response detectable by means of the sensing arrangement.

According to an aspect a connector for a coupling assembly for fluidically connecting an inflatable/deflatable article 120 and a pump 110 in a fluid pressure control system 100 is provided. The connector may be a connector according to any previously described embodiments.

Thus, the connector 330, 630 may be connectable to the connecting member 310, 610 to upon engagement form a fluid pathway through said connector 330, 630 and connecting member 310, 610 by means of insertion of a distal part 331, 631 of the connector 330, 630 into the connecting member 310, 610.

The distal part 331, 631 may be movable inside the connecting member 310, 610 along a connection axis CA extending distally from the connector 330, 630 between a coupled position and a non-coupled position,

The connector 330, 630 may be provided with a position indicating part 390, 490, 690, whereby the position of the position indicating part 390, 490, 690 is detectable by means of a sensing arrangement 320 during engagement between the connector 330, 630 and the connecting member 310, 610.

The position indicating part 390, 490, 690 is detectable by means of a sensor unit 321, 421, 621 of the sensing arrangement 320. The sensor unit 321, 421, 621 may be provided on the connecting member 310, 610.

A central point of the position indicating part 390, 490, 690 may be arranged to be at a distance dc along the connection axis CA in a distal direction from a center point of a sensor unit 321, 421, 621 of the sensing arrangement 320 when the distal part 331, 631 is in the coupled position. In one embodiment, the distance dc may be between 1 mm and 0.01 mm. In one embodiment, the distance dc may be preferably less than 0.5 mm.

The position indicating part 390, 490, 690 may be provided on the distal part 331, 631 of the connector 330, 630.

The position indicating part 390, 490 may be made of a material selected from a group consisting of a ferrite material, steel and a brass material.

The position indicating part 690 may comprise any one of the following: a reflective outer material, an uneven contour or texture, polished surface, an indicating color.

The position indicating part 390, 490, 690 may be in the form of a position indicating part, said position indicating part being adapted to generate a characteristic response associated with the inflatable/deflatable article 120 or the pump 110.

The characteristic response may be detectable by means of the sensing arrangement 320 for identifying the inflatable/deflatable article 120 or the pump 110.

According to an aspect, an inflatable/deflatable article arrangement for connection to a pump 110 in a fluid pressure control system 100 by means of a coupling assembly 300, 600 is provided. The inflatable/deflatable article arrangement comprising an inflatable/deflatable article 120 and a connector according to any one of previously described embodiments fluidically connected to the inflatable/deflatable article 120.

According to an aspect, a method for monitoring a coupling assembly for fluidically connecting an inflatable/deflatable article and a pump of a fluid pressure control system is provided. The fluid pressure control system comprises a controller operatively connected to the pump for controlling said pump. The coupling assembly comprises a connector and a connecting member. The connector is connectable to the connecting member to form a fluid pathway through said connector and connecting member by means of insertion of a distal part of the connector into the connecting member. The distal part is movable inside the connecting member along a connection axis extending distally from the connector between a coupled position and a non-coupled position, wherein the coupling assembly comprises a position indicating arrangement comprising a position indicating part provided on the connector or the connecting member and a sensing arrangement.

The method comprises detecting the position of the position indicating part provided on the connector or the connecting member relative the other of the connector or the connecting member by means of the sensing arrangement.

The method further comprises generating a position signal to the controller indicative of the position of the distal part relative to the connecting member. The position signal is based on the detected position of the position indicating part.

In one embodiment, the method may further comprise providing on an indicating device operatively connected to the controller, an indication to a user based on the position signal.

In one embodiment, the method may further comprise providing an indication to a user in response to the distal part being in the coupled position and/or the non-coupled position.

The method may further comprise providing an indication to a user in response to the distal part being in an intermediate position along the connection axis between the coupled position and the non-coupled position.

The indicating device may be configured to provide an indication to a user within a time interval in response to the distal part being moved from a non-inserted position to the non-coupled position. The time interval is preferably smaller than 5 seconds.

The coupling assembly may further comprise identifying means provided on the connector or the connecting member. The component identifying means serves to identify the inflatable/deflatable article or the pump. The method may further comprise detecting a characteristic response generated by the identifying means.

In one embodiment, the method comprises detecting a characteristic response generated by the identifying means while the distal part is inserted into the connecting member.

Further, the method may comprise comparing the response with a set of stored responses associated with a corresponding set of pumps or inflatable/deflatable articles to identify the pump or inflatable/deflatable article.

In one embodiment, the method may further comprise deactivating the position indicating arrangement in response to the comparison with the set of stored responses.

In one embodiment, the method may further comprise detecting the position of the position indicating part by obtaining a measured value from the sensing arrangement and generating a position signal based on a measured value associated with a detected transient maximum when the distal part is between the non-coupled position and the coupled position and a position signal associated with a detected lower steady state when the distal part is in the coupled position.

In one embodiment, the method may further comprise controlling the pump based on the position signal.

In one embodiment, the pressure provided by the pump 110 is controlled based on the position signal such that the pressure provided by the pump 110 is dependent on the position of the connector 330, 630 relative to the connecting member.

Thus, the method may comprise controlling the pressure provided by the pump 110 based on the position of the connector relative to the connecting member.

The invention has been described above in detail with reference to embodiments thereof. However, as is readily understood by those skilled in the art, other embodiments are equally possible within the scope of the present invention, as defined by the appended claims.

1-71. (canceled)

72. A coupling assembly for fluidically connecting an inflatable/deflatable article and a pump in a fluid pressure control system, the coupling assembly comprising a connector and a connecting member, the connector being connectable to the connecting member to upon engagement form a fluid pathway through said connector and connecting member, the coupling assembly further comprising a position indicating arrangement, said position indicating arrangement comprising a position indicating part provided on the connector or the connecting member and a sensing arrangement for detecting the position of the position indicating part relative said sensing arrangement during engagement between said connector and connecting member.

73. The coupling assembly according to claim 72, wherein the position indicating part is provided on the connector or the connecting member and the sensing arrangement is at least partly provided on the other of the connector or the position indicating part,
wherein the connector is connectable to the connecting member to form a fluid pathway through said connector and connecting member by means of insertion of a distal part of the connector into the connecting member, said distal part being movable inside the connecting member along a connection axis (CA) extending distally from the connector between a coupled position and a non-coupled position,
wherein the position indicating arrangement is configured to generate a position signal to a controller of the fluid pressure control system indicative of the position of the distal part relative to the connecting member, whereby the position signal is based on the position of the position indicating part provided on the connector or the connecting member relative the other of the connector or the connecting member, and
wherein the position signal is associated with a corresponding degree of insertion of the distal part into connecting member.

74. The coupling assembly according to claim 72, wherein the position signal is based on a measured value obtained by the sensing arrangement said measured value being a detected transient maximum when the distal part is between the non-coupled position, the coupled position, and a detected lower steady state when the distal part is in the coupled position.

75. The coupling assembly according to claim 72, wherein the sensing arrangement comprises at least one sensor unit.

76. The coupling assembly according to claim 72, wherein a central point of the position indicating part is arranged to be at a distance (dc) along the connection axis (CA) in a distal direction from a center point of a sensor unit of the sensing arrangement when the distal part is in the coupled position.

77. The coupling assembly according to claim 72, wherein the sensor unit is an IR-sensor unit or a visible light sensing unit such as a LED-based sensor unit.

78. The coupling assembly according to claim 72, wherein the position indicating part is provided in a proximal position of the connector or the connecting member such that said position indicating part moves along a connection axis (CA) when the distal part moves distally between non-coupled position and the coupled position.

79. A connector for a coupling assembly for fluidically connecting an inflatable/deflatable article and a pump in a fluid pressure control system, wherein the connector is connectable to the connecting member to upon engagement form a fluid pathway through said connector and connecting member by means of insertion of a distal part of the connector into the connecting member said distal part being movable inside the connecting member along a connection axis (CA) extending distally from the connector between a coupled position and a non-coupled position, and
wherein the connector is provided with a position indicating part whereby the position of the position indicating part is detectable by means of a sensing arrangement during engagement between the connector and the connecting member.

80. The connector according to claim 79, wherein a central point of the position indicating part is arranged to be at a distance (dc) along the connection axis (CA) in a distal direction from a center point of the sensor unit of the sensing arrangement when the distal part is in the coupled position.

81. The connector according to claim 79, wherein the characteristic response is detectable by means of the sensing arrangement for identifying the inflatable/deflatable article or the pump.

82. A compression therapy system comprising an inflatable/deflatable article, a pump and a controller operatively connected to the pump, the compression therapy system further comprising a coupling assembly for fluidically connecting the inflatable/deflatable article and the pump, the coupling assembly comprising a connector and a connecting member, the connector being connectable to the connecting member to upon engagement form a fluid pathway through said connector and connecting member,
the coupling assembly further comprising a position indicating arrangement, said position indicating arrangement comprising a position indicating part provided on the connector or the connecting member and a sensing arrangement for detecting the position of the position indicating part relative said sensing arrangement during engagement between said connector and connecting member.

83. The compression therapy system according to claim 82, wherein the position indicating part is provided on the connector or the connecting member and the sensing arrangement is at least partly provided on the other of the connector or the position indicating part.

84. The compression therapy system according to claim 82, wherein the connector is connectable to the connecting member to form a fluid pathway through said connector and connecting member by means of insertion of a distal part of the connector into the connecting member, said distal part being movable inside the connecting member along a connection axis (CA) extending distally from the connector between a coupled position and a non-coupled position.

85. The compression therapy system according to claim 82, wherein the position signal is based on a measured value obtained by the sensing arrangement, said measured value being associated with a detected transient maximum when the distal part is between the non-coupled position and the coupled position and a detected lower steady state when the distal part is in the coupled position.

86. The compression therapy system according to claim 82, wherein the position signal is based on a measured value obtained by the sensing arrangement said measured value being a detected transient maximum when the distal part is between the non-coupled position and the coupled position and a detected lower steady state when the distal part is in the coupled position.

87. The compression therapy system according to claim 82, wherein the indicating device is provided on the pump.

88. The compression therapy system according to claim 82, wherein the indicating device is configured to provide an indication to a user in response to the distal part being in the coupled position and/or the non-coupled position, and
wherein the indicating device is configured to provide an indication to a user in response to distal part being in an intermediate position along the connection axis (CA) between the coupled position and the non-coupled position.

89. The compression therapy system according to claim 82, wherein the coupling assembly further comprises component identifying means provided on the connector or the connecting member, said component identifying means serving to identify the inflatable/deflatable article, and
wherein the position indicating part is in the form of an identification component, said identification component being adapted to generate a characteristic response associated with the inflatable/deflatable article.

90. The compression therapy system according to claim 89, wherein the characteristic response is detectable by means of the sensing arrangement or a separate component sensor operatively connected to the controller, whereby the controller is configured to compare the characteristic response with a set of stored characteristic responses associated with a corresponding set of inflatable/deflatable articles to identify the inflatable/deflatable article.

91. The compression therapy system according to claim 90, wherein the controller is configured to selectively deactivate the position indicating arrangement in response to the comparison with the set of stored characteristic responses.
